(19) United States
(12) Patent Application Publication
(10) Pub. No.: US 2018/0179489 A1
(43) Pub. Date: Jun. 28, 2018

(54) DIGESTIBLE SUBSTRATES FOR CELL CULTURE

(71) Applicant: Corning Incorporated, Corning, NY (US)

(72) Inventors: Stephen Joseph Caracci, Elmira, NY (US); David Henry, Fontaine le Port (FR); Corinne Walerack, Veneux les sablons (FR); Yue Zhou, Horseheads, NY (US)

(21) Appl. No.: 15/579,739

(22) PCT Filed: Jun. 8, 2016
(86) PCT No.: PCT/US2016/036371

§ 371 Date: Dec. 5, 2017

Related U.S. Application Data
(60) Provisional application No. 62/233,044, filed on Sep. 25, 2015.
(60) Provisional application No. 62/172,299, filed on Jun. 8, 2015.

(51) Int. Cl. C12N 5/00; C12N 5/0775; C12N 5/071

(52) U.S. Cl. CPC C12N 5/0075; C12N 5/0663; C12N 5/0686; C12N 2533/18; C12N 2531/00; C12N 2533/70; C12N 5/0688

(57) ABSTRACT

A cell culture article is provided. The cell culture substrate includes a polygalacturonic acid compound selected from at least one of: pectic acid or salts thereof, and partially esterified pectic acid having a degree of esterification from 1 to 40 mol % or salts thereof. The polygalacturonic acid compound is crosslinked with a divalent cation and the divalent cation concentration ranges from 0.5 to 2 g/1 of the substrate.

### CROSS-REFERENCE TO RELATED APPLICATIONS

This application claims the benefit of priority under 35 U.S.C. § 119 of U.S. Provisional Application Ser. Nos. 62/233,044 filed on Sep. 25, 2015 and 62/172,299 filed on Jun. 8, 2015, the contents of which are relied upon and incorporated herein by reference in their entirety. This application is related to commonly-assigned U.S. Pat. Nos. 8,4044,85 and 8,426,176 and to co-pending and commonly-assigned International Application Nos. WO2014/209865 and WO2014/0120616, the contents of which are relied upon and incorporated herein by reference in their entirety.

### BACKGROUND

### Field

The present disclosure relates generally to methods of making digestible substrates, and more specifically to transparent, digestible microcarriers that may be used, by way of example, for the isolation of proteins, cells, and viruses and also for diagnostic applications and cell cultivation.

### Technical Background

In contrast to cell culture on flat surfaces where adhesive cells can reach high confluence and thus limit cell expansion via cell-to-cell contact inhibition, spherically-shaped microcarriers having a high ratio of surface area/volume present an attractive platform for efficient cell culture scale-up or expansion where either harvested cells or conditioned media can be the desired product.

Incumbent to cell culture is adequate oxygenation and supply of nutrients to the cells. An associated challenge includes stirring of the microcarriers to provide the required oxygen and nutrients without introducing hydrodynamic stresses sufficient to damage the growing cells. Conventionally the stirring is done using impellers.

A further challenge involves separating the microcarriers from the cells or conditioned media. Enzymatic treatment may be used to harvest adhesive cells, for example, though the addition of enzymes can damage the cells. Proteolytic enzymes, for example, may non-selectively clear cell surface receptors.

Trypsin is frequently applied to dissociate adhesive cells from the substratum once cultured cells reach confluence. As an example, in a method for culturing and harvesting anchorage-dependent cells employing microcarrier beads coated with collagen, once growth is complete the collagen may be digested off of the microcarrier. However, due to the proteolytic activity of trypsin, cell surface proteins are often cleaved, which may lead to unwanted disruption of cell function. It is believed that trypsin induces proteome alteration and cell physiological changes. Trypsinization may induce down-regulated growth- and metabolism-related protein expressions and up-regulated apoptosis-related protein expressions, implying that trypsin used for cell subculture may have an adverse effect on cell physiology.

As another example of the deleterious effect of proteases, it is also known that treatment of cells with proteases such as trypsin remove antigens from cancer cells and thus might render them unusable to develop vaccines for anti-cancer therapies. As a consequence, harvesting cells without trypsin or in the absence of proteolytic enzymes such as trypsin is highly desirable.

In view of the foregoing, it would be advantageous to provide a low-cost, efficient approach to synthesize cell growth surfaces including microcarriers having, inter alia, a controlled particle size, composition, uniformity and crystalline structure, as well as a surface chemistry supportive of cell attachment and/or growth that enable non-proteolytic cell separation and harvesting.

### BRIEF SUMMARY

In accordance with embodiments of the present disclosure, a cell culture article comprises a substrate that includes a polygalacturonic acid compound selected from at least one of pectic acid or salts thereof and partially esterified pectic acid having a degree of esterification from 1 to 40 mol % or salts thereof. The polygalacturonic acid compound is crosslinked with a divalent cation. The divalent cation concentration in the substrate ranges from 0.5 to 2 g/l of the substrate.

A method of making a cell culture article comprises dispensing (i.e., dropwise) a hydrocolloid solution into a gelation bath. The hydrocolloid solution comprises a polygalacturonic acid (PGA) compound selected from at least one of pectic acid or salts thereof, and partially esterified pectic acid having a degree of esterification from 1 to 40 mol % or salts thereof. The gelation bath comprises a divalent metal salt.

A method for culturing cells comprises contacting cells with a cell culture medium having a cell culture article as described above and culturing the cells in the medium. A method for harvesting cultured cells comprises culturing cells on the surface of the cell culture described above and contacting the cultured cells with a mixture of pectinase and a chelator to separate the cells from the article.

In embodiments, the microcarriers are transparent, enabling cell observation, and are suitable for large-scale cell propagation in chemically-defined medium or serum-supplemented medium.

Preparation of the PGA substrates is compatible with existing high throughput manufacturing processes enabling the preparation of microcarriers having a uniform size distribution. The uniform size distribution eliminates the need for an extra sieving step, which is labor intensive, time consuming, and requires additional equipment and large volumes of water and organic solvent. The presently-disclosed synthesis is not based on an emulsion process and therefore does not require the use of a surface-active agent or a high volume of organic solvents as a dispersion medium. Further, the method uses water soluble forms of calcium and therefore enables the preparation of homogeneous and transparent PGA substrates having a smooth surface that allows easy cell observation. The method is environmentally friendly and more economical than approaches based on emulsification or internal gelation.

Digestible cell culture articles are disclosed in commonly-assigned International Publication Number WO2014/209865, the contents of which are incorporated herein by reference in their entirety.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIGS. 1A, B and C are illustrations of different sizes of microcarriers according to embodiments;

FIG. 2 is a graph showing calcium content as a function of PGA concentration;

FIG. 3 is a phase contrast image showing hMSC cells on externally crosslinked PGA microcarriers according to an embodiment;

FIG. 4 is a phase contrast image showing hMSC cells on externally crosslinked PGA microcarriers according to a further embodiment;

FIG. 5 is a phase contrast image showing hMSC cells on externally crosslinked PGA microcarriers according to a still further embodiment;

FIG. 6 shows phase contrast images showing MRC5 and Vero cells on gelatin-coated PGA microcarriers according to an embodiment;

FIG. 7 is a graph of fold expansion for Vero cells on gelatin-coated dextran microcarriers and on gelatin-coated externally crosslinked microcarriers;

FIG. 8 is a graph of fold expansion for MRC5 cells on gelatin-coated dextran microcarriers and on gelatin-coated externally crosslinked microcarriers;

FIG. 9 is a graph of fold expansion for hMSC cells on both gelatin-coated digestible microcarriers and microcarriers provided with a Corning Incorporated Synthemax® II surface;

FIG. 10 is a phase contrast image showing monodisperse PGA beads made according to an embodiment;

FIG. 11 is a phase contrast image showing MRC5 cells on externally crosslinked PGA microcarriers according to an embodiment;

FIG. 12 is a phase contrast image showing comparative microcarriers illustrating the broad size distribution obtain by emulsification and internal gelation; and

FIG. 13 is a phase contrast image of hMSC cells in serum-free media after seeding on VN-grafted PGA microcarriers.

FIGS. 14A, C and E are microscopic images of size-controlled microcarriers according to embodiments, FIGS. 14B, D and F show the size distribution of the size-controlled microcarriers shown in FIGS. 14A, C and E.

FIG. 15 is a drawing of a cuvette, in embodiments.

FIG. 16 is an illustration of settling of microcarriers out of solution including a graph illustrating the OD of a standard settling process.

FIG. 17 is a graph illustrating settling times of exemplary microcarriers, according to embodiments, compared to prior art products.

### DETAILED DESCRIPTION

Reference will now be made in greater detail to various embodiments of the subject matter of the present disclosure, some embodiments of which are illustrated in the accompanying drawings. The same reference numerals will be used throughout the drawings to refer to the same or similar parts.

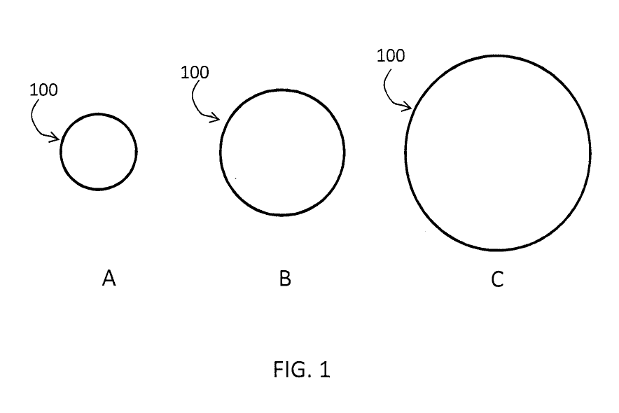

Disclosed are cell culture articles that promote cell attachment and growth and which allow for cell harvesting without the use of protease. Example cell culture articles are microcarriers, which are also referred to as beads or microbeads (collectively “microcarriers”). FIGS. 1A, B and C, illustrate three different sizes of microcarrier beads, 100, in embodiments. In embodiments, the cell culture article is a smooth and transparent (or translucent) bead comprising a gel that includes pectic acid, partially esterified pectic acid, or salts thereof. The cell culture articles may be spherical or substantially spherical and are formed by external gelation. The calcium content of the cell culture articles may be adjusted to afford rapid cell harvesting under mild conditions that mitigates damage to the cells. Molecules promoting the attachment of anchorage-dependent cells may be attached to the surface of the cell culture article by chemical coupling or physical adsorption.

In contrast to the presently-disclosed external gelation route, microcarriers may be formed via emulsification and internal gelation. With this internal gelation process, beads are formed via gelation of a PGA aqueous solution containing an insoluble calcium salt dispersed in the aqueous phase, which is emulsified within an oil phase (also called a continuous phase or dispersion medium).

In the case of internal gelation, crosslinking is initiated by addition of an oil-soluble acid that releases soluble divalent metal ions (e.g., Ca²⁺ or Mg²⁺) from the salt. With such a method, however, a large volume of the oil phase is required as is a significant amount of surfactant to stabilize the emulsion. While vegetable oils can be used as the continuous phase, beads prepared in this dispersion medium are difficult to rinse.

A further drawback to the internal gelation process is that a portion of the metal ion source (salt) may remain intact and manifest as heterogeneities in the microcarriers, which may compromise surface roughness and transparency. Further, such retained metal salt may be released over time during use of the microcarriers, which may be detrimental to cell culture or inhibit digestion of the microcarriers during cell harvest.

The disclosed external gelation methods provide an inexpensive and environmentally-friendly synthetic route for the preparation of highly-transparent PGA microcarriers that are free of undesired inclusions (second phases) and surface defects and which support non-proteolytic cell separation and harvesting. As defined herein, transparent microcarriers exhibit at least 90% transmission over the visible spectrum, i.e., 90, 92, 94, 96, 98, 99 or 100% transmission, including ranges between any of the foregoing values, from 390 to 700 nm.

In addition, in embodiments, uniform size distribution of the microcarriers can be provided. Uniform size distribution ensures faster and cleaner separation of microcarriers from supernatant during use. This can make medium exchange and final production isolation more predictable, more reliable, and less expensive. In embodiments, microcarrier size can be precisely tuned to different ranges. This allows the settling speed of the beads to be customized to match different bioprocess needs without changing the material properties of the beads

PGA Polymers

Microcarriers may be made using at least one ionotropically crosslinked polysaccharide. Examples include pectic acid, also known as polygalacturonic acid (PGA), or salts thereof, or partly esterified pectic acid (PE PGA) known as pectinic acid, or salts thereof.

Pectic acid can be formed via hydrolysis of certain pectin esters. Pectins are cell wall polysaccharides and in nature have a structural role in plants. Major sources of pectin include citrus peel (e.g., peels from lemons and limes) and apple peel. Pectins are predominantly linear polymers based on a 1,4-linked alpha-D-galacturonate backbone, interrupted randomly by 1,2-linked L-rhamnose. The average molecular weight ranges from about 50,000 to about 200,000 Daltons.

The polygalacturonic acid chain of pectin may be partly esterified, e.g., with methyl groups and the free acid groups may be partly or fully neutralized with monovalent ions such as sodium, potassium, or ammonium ions. Polygalacturonic acids partly esterified with methanol are called pectinic acids, and salts thereof are called pectinates. The degree of methylation (DM) for high methoxyl (HM) pectins can be, for example, from 60 to 75 mol % and those for low methoxyl (LM) pectins can be from 1 to 40 mol %.

In embodiments where pectinic acid is selected, the degree of esterification may be 40 mol % or less (e.g., 1, 5, 10, 20, 30 or 40 mol %, including ranges between any of the foregoing values). Higher degrees of esterification make bead formation by ionotropic crosslinking ineffective. Without being bound by theory, it is believed that a minimum amount of free carboxylic acid groups (not esterified) are needed to obtain a desirable degree of ionotropic crosslinking.

In embodiments, microcarrier beads were formed using LM pectins such as polygalacturonic acid that contains 20 mol % or less of methoxyl groups, e.g., 0, 5, 10, 15 or 20 mol %. Such a polygalacturonic acid may have no or negligible methyl ester content as pectic acids. As used herein, pectinic acid having no or only negligible methyl ester content and low methoxyl (LM) pectins are referred to collectively as PGA.

In embodiments, microcarrier beads were formed using a mixture of pectic acid and pectinic acid. Pure pectic acid and/or pectinic acid may be used. Blends with compatible polymers may also be used. For example, pectic or pectinic acid may be mixed with polysaccharides such as dextran, substituted cellulose derivatives, alginic acid, starches, glycogen, arabinoxylans, agarose, etc. Glycosaminoglycans like hyaluronic acid and chondroitin sulfate, or various proteins such as elastin, fibrin, silk fibroin, collagen and their derivatives can be also used. Other water soluble synthetic polymers can be also blended with pectic acid and/or pectinic acid. Non-limiting examples include polyalkylene glycol, poly(hydroxyalkyl(meth)acrylates), poly(meth)acrylamide and derivatives, poly(N-vinyl-2-pyrrolidone), polyvinyl alcohol, etc. Compatible polymers may be anionic, neutral or cationic provided that their inclusion does not impair digestion of the microcarriers.

External Ionotropic Gelation

External gelation, also called diffusion setting, involves the introduction of a hydrocolloid (PGA) solution to an ionic solution, with gelation occurring via diffusion of ions into the hydrocolloid solution. In embodiments, an aqueous, negatively-charged polysaccharide solution was dispensed drop-wise into a solution of divalent cations, such as calcium, magnesium or barium, which induces crosslinking of the PGA polymer. The crosslinking is ionic crosslinking, which in contrast to covalent crosslinking allows for subsequent digestion of the crosslinked polymer.

According to embodiments, the PGA concentration in the hydrocolloid solution ranged from 0.5 to 5 wt. %, e.g., 0.5, 1, 1.5, 2, 2.5, 3, 3.5, 4, 4.5 or 5 wt. %, including ranges between any of the foregoing values.

Example methods for forming droplets of the hydrocolloid (PGA) solution included dripping or extrusion with a syringe; jet breakup or pulverization, for which bead formation is accomplished by a coaxial air stream that pulls droplets from a nozzle; electrostatic bead generation, which uses an electrostatic field to pull droplets from a nozzle into a gelling bath; magnetically driven vibration; jet cutting, for which bead formation is accomplished by a rotating cutting tool that cuts a jet into uniform cylindrical segments; and spinning disk atomization.

Droplets of the PGA solution may be spherical or substantially spherical and have an average diameter ranging from 10 to 500 micrometers, e.g., 10, 20, 25, 50, 75, 100, 150, 200, 252, 300, 350, 400, 450 or 500 micrometers, including ranges between any of the foregoing values.

The gelling bath may comprise an aqueous solution of a divalent metal salt. In embodiments, the salt (e.g., calcium chloride) concentration in the gelling bath is at least 1% (w/v), e.g., 1, 2, 4, 6, 8, 10, 12, 14, 16, 18 or 20%, including ranges between any of the foregoing values. If the calcium content is too low, the beads exhibit poor stability due to a too low crosslinking density.

The aqueous solution may comprise an alcohol such as ethanol. The ratio (v/v) of alcohol to water may range from 0/100 to 80/20, e.g., 0/100, 10/90, 20/80, 30/70, 40/60, 50/50, 60/40, 70/30 and 80/20.

In embodiments, some covalent crosslinking can occur but the level of such crosslinking, being irreversible, should be sufficiently low, for example, less than about 10 to 20 mol %, so as to maintain the digestibility of the beads.

The microcarrier beads may be spherical or substantially spherical and have an average diameter ranging from 10 to 500 micrometers, e.g., 10, 20, 25, 50, 75, 100, 150, 200, 250, 300, 350, 400, 450 or 500 micrometers, including ranges between any of the foregoing values. In embodiments, the coefficient of variation (CV) of the microcarrier beads, also referred to as the relative standard deviation, is less than 20%, e.g., 2, 5, 10 or 15%, including ranges between any of the foregoing. In embodiments, the size spread Δd5-d95 (the difference between d95 and d5, where d5 is the microcarrier diameter that is larger than the diameters of 5% of the microcarrier population and d95 is the microcarrier diameter that is larger than the diameters of 95% of the microcarrier population) is less than 25 micrometers, e.g., 10, 15 or 20 micrometers, including ranges between any of the foregoing. In embodiments, the size spread Δd10-d90 (the difference between d90 and d10, where d10 is the microcarrier diameter that is larger than the diameters of 10% of the microcarrier population and d90 is the microcarrier diameter that is larger than the diameters of 90% of the microcarrier population) is less than 20 micrometers, e.g., 5, 10 or 15 micrometers, including ranges between any of the foregoing. In embodiments, the radius of curvature spread from d5 to d95 (the difference between the radius of curvature of the microcarrier diameter that is larger than the diameters of 5% of the microcarrier population and the radius of curvature of the microcarrier diameter that is larger than the diameters of 95% of the microcarrier population) is less than 10 cm⁻¹, e.g., 2, 5 or 8 cm⁻¹, including ranges between any of the foregoing.

Control of Bead Size

In embodiments, microcarrier beads can be manufactured within narrow and specific size ranges. That is, they can be size-controlled. Control of the size of microcarrier beads is important for several reasons. If there is a wide size distribution, ranging from small to large microcarriers, microcarriers with smaller size will be in suspension much longer than larger size microcarriers. Exact settling time in the process would be much longer (because of the presence of smaller beads) or difficult to define. In use, more time will be required to ensure that the supernatant is clear from microcarriers.

Narrow size distribution enables settling of beads at a consistent speed which allows for more predictable separation of microcarriers from supernatant during medium exchange or culture produce isolation. Size of microcarriers can be fine-tuned to different ranges to control the settling speed. This enables customization of settling speed to match different process needs. Size controlled microcarriers have uniform surface area, which provides the same area available for cells to seed per microcarrier. This makes calculating the surface area available for cell seeding easier. In addition, cells will reach confluence at the same, or at a similar, time. As used herein, the terms “confluence” or “confluent” are used to indicate when cells have formed a coherent layer on a growth surface where all cells are in contact with other cells, so that virtually all the available growth surface is used. For example, “confluent” has been defined (R. I. Freshney, Culture of Animal Cells—A Manual of Basic Techniques, Second Edition, Wiley-Liss, Inc. New York, N.Y., 1987, p. 363) as the situation where “all cells are in contact all around their periphery with other cells and no available substrate is left uncovered”. As is conventional, the amount of a growth surface that is covered by cells is referred to as a proportion of confluence. For example, a situation where approximately half of the growth surface is covered by cells is referred to herein as 50% confluence, or, in the alternative, as half confluence. Size-controlled microcarriers can be suspended in the same agitation conditions. This enables fine control of shear force to balance good suspension of microcarriers and may allow conditions that cause less damage to cells. Well defined settling times for different groups of size-controlled microcarriers can help easy separation during continuous cell culture to prevent uneven cell growth on beads fed at different times. For example, cells can be seeded on size-controlled microcarriers with 250 μm size first. After cells have reached half confluence, size-controlled microcarriers with of 350 μm size can be added in the bioreactor for bead-to-bead transfer. At the time of confluence for 250 μm microcarriers, microcarriers with this size can be removed by their unique settle speed or by filtration. Only beads with 350 μm size and half confluent are left in the bioreactor. Then, fresh 250 μm microcarriers can be added. After 350 μm microcarriers reach confluence, they can be collected and fresh 350 μm microcarriers added. This process may ensure that all the beads are removed when they reach confluence. In contrast, where microcarriers of the same size are used to do bead-to-bead transfer and continuous cell culture, cells on the beads from an earlier feeding will stay in bioreactor much longer than those on beads from a later feeding and the quality of cells can be deteriorated as a result of over confluence.

In embodiments, dissolvable microcarriers were size-controlled during manufacture using a vibration encapsulator. Size-controlled beads were formed by going through a nozzle with defined hole size, flow rate and vibration frequency. The size of obtained beads was controlled to a narrow range with a coefficient of variation of less than 10%.

Bead Digestion

Non-proteolytic enzymes suitable for digesting the microcarrier, harvesting cells, or both, include pectinolytic enzymes or pectinases, which are a heterogeneous group of related enzymes that hydrolyze the pectic substances.

Cell harvesting involves contacting cell-laden microcarriers with a solution comprising a mixture of pectinolytic enzyme or pectinase and a divalent cation chelating agent.

An example method for harvesting cultured cells comprises culturing cells on the surface of a microcarrier as disclosed herein, and contacting the cultured cells with a mixture of pectinase and a chelator to separate the cells from the microcarrier.

Pectinases (polygalacturonase) are enzymes that break down complex pectin molecules to shorter molecules of galacturonic acid. Pectinases catalyze the liberation of pectic oligosaccharides (POS) from polygalacturonic acid. Pectinases are produced by fungi, yeast, bacteria, protozoa, insects, nematodes and plants. Commercially-available sources of pectinases are generally multi-enzymatic, such as Novozyme Pectinex™ ULTRA SPL, a pectolytic enzyme preparation produced from a selected strain of Aspergillus aculeatus. Novozyme Pectinex™ ULTRA SPL contains mainly polygalacturonase, (EC 3.2.1.15) pectintranseliminase (EC 4.2.2.2) and pectinesterase (EC: 3.1.1.11). The EC designation is the Enzyme Commission classification scheme for enzymes based on the chemical reactions the enzymes catalyze. Pectinases are known to hydrolyze pectin. They may attack methyl-esterified pectin or de-esterified pectin.

The concentration of pectinolytic enzyme in the digestion solution may be 1 to 200 U, e.g., 1,2, 5, 10, 20, 50, 100, 150 or 200 U, including ranges between any of the foregoing.

Example chelating agents include ethylenediaminetetraacetic acid (EDTA), cyclohexanediaminetetraacetic (CDTA), ethylene glycol tetraacetic acid (ETGA), citric acid, tartaric acid, etc. The chelating agent concentration in the digestion solution may be 1 to 200 mM, e.g., 10, 20, 50, 100, 150 or 200 mM. To prevent cytotoxic side effects, the concentration of chelating agent in the digestion solution may be 10 mM or less, e.g., 1, 2, 5, or 10 mM, including ranges between any of the foregoing.

In embodiments, the total volume of the digestion solution comprising the pectinolytic enzyme and the chelating agent is less than 10 times the microcarrier volume, e.g., 1, 2, 4, 5 or 10 times the volume of the microcarriers including ranges between any of the foregoing values.

Depending of the digestion time, temperature, and amount of pectinolytic enzyme added, the extent of digestion beads can be selected or predetermined. It has been observed that cells detach from the microcarrier surface before the bead is fully digested. It is therefore possible to harvest cells with or without complete digestion of the beads. In embodiments where cells are harvested from partially-digested microcarriers, separation of the cells from remnant microcarriers may be done by one or more of filtration, decantation, centrifugation, and like processing.

Beads are readily digested when their calcium content is less than 2 g/l of moist beads, e.g., less than 2, 1.5, 1, 0.8 or 0.5 g/l. When the calcium content of the beads at the harvest stage is greater than 1 g/l, a greater volume and/or concentration of pectinolytic enzyme and divalent cation chelating agent can be used. The time for complete digestion may be less than one hour, e.g., 10, 15, 30 or 45 min. As used herein, the term “complete digestion” refers to digestion of microcarriers that results in a microcarrier particle count that complies with the particle count test as described in The United States Pharmacopeia and The National Formulary Section 788 (USP<788>) entitled “Particulate Matter in Injections”. As indicated in USP<788> a preparation complies with the test if the average number of particles present in the units tested does not exceed 25 particles per mL equal to or greater than 10 μm and does not exceed 3 particles per mL equal to or greater than 25 μm. In embodiments, the microcarrier particle count for particles having a size of greater than or equal to 10 μm after digestion of the microcarriers is less than 10 particles, e.g., 0, 1, 2, 3, 4, 5, 6, 7, 8 or 9, including ranges between any of the foregoing. In embodiments, the microcarrier particle count for particles having a size of greater than or equal to 25 μm after digestion of the microcarriers is less than 1 particle, e.g., 0, 0.1, 0.2, 0.3, 0.4, 0.5, 0.6, 0.7, 0.8 or 0.9, including ranges between any of the foregoing.

As defined herein the “moist bead” volume is the volume of the bed of beads after decantation or centrifugation. The bed comprises swollen beads as well as interstitial water (i.e., water present between the swollen beads). According to measurements, moist beads contain 70 vol. % swollen beads and 30 vol. % interstitial water. The swollen beads contain 99% water for a 1% PGA solution, 98% water for a 2% PGA solution, 97% water for a 3% PGA solution, etc.

By way of example, microbeads prepared from a 3% (w/v) PGA sol contain, at equilibrium, about 1.48 g/l calcium ions. Complete digestion of the microbeads in less than 10 minutes results from exposure to at least 10 mM EDTA and at least 50 U enzyme using a 5× volume of digestion solution (compared to the volume of beads).

Cell Attachment

PGA beads, due to their hydro gel nature and negative charge, do not readily support cell attachment without specific treatment. In order to promote attachment of anchorage dependent cells, the microbeads can be provided with a coating or other surface treatment. By way of example, the PGA beads can be functionalized with moieties promoting cell adhesion, for example, peptides such as those comprising a RGD sequence.

Further candidate peptides include those containing amino acid sequences potentially recognized by proteins from the integrin family, or leading to an interaction with cellular molecules able to sustain cell adhesion. Examples include BSP, vitronectin, fibronectin, laminin, Type I and IV collagen, denatured collagen (gelatin), and like peptides, and mixtures thereof. Further example peptides are BSP and vitronectin (VN) peptides having the following sequences: Ac-Lys-Gly-Pro-Gln-Val-Thr-Arg-Gly-Asp-Val-Phe-Thr-Met-Pro-NH₂ (seq. ID No. 1), and Ac-Lys-Gly-Gly-Asn-Gly-Glu-Pro-Arg-Gly-Asp-Thr-Tyr-Arg-Ala-Tyr-NH₂ (seq. ID No. 2), respectively.

In embodiments, the microbeads are surface functionalized with cell adhesion promoting recombinant proteins, which can be grafted or applied as a coating. Example recombinant proteins include fibronectin-like engineered proteins marketed under the trade names ProNectin® and ProNectin® plus, though other recombinant proteins that promote attachment of anchorage dependent cells can be used.

### EXAMPLES

### Example 1

### 1% PGA Microbeads Crosslinked with 3% Calcium

Microbeads were prepared from a 1 wt. % solution of polygalacturonic acid (PGA) by dissolving polygalacturonic acid sodium salt (Sigma catalog number #P3850) into water at 80-85° C. under constant agitation. The solution was filtered using a 20 micrometer polypropylene filter under vacuum to eliminate particles in suspension.

A gelling bath was produced in a separate beaker using 400 ml of a 3% w/v calcium chloride water/ethanol (75/25 v/v) solution, which was stirred using a magnetic stirrer.

Droplets were produced via the addition of 25 ml of the PGA solution to the gelling bath using a syringe equipped with a 30 Gauge needle. A syringe pressure of about 2 bars was applied.

Beads were hardened in the calcium chloride bath for 120 minutes before being washing four times with water. The calcium content within the beads was determined as described in example 9. After four rinses, the calcium concentration was about 0.5-0.6 g/l of moist beads.

The beads were stored in sterile water in sterile containers at 4° C. prior to coating. The beads were highly transparent without any observable surface defects.

When contacted with 5 mM EDTA/50 U pectinase at 25° C., the beads dissolved completely within 5 minutes.

### Example 2

### 1% PGA Microcarriers Crosslinked with 12% Calcium

The procedure from example 1 was repeated except that the 3% w/v calcium chloride water/ethanol (75/25 v/v) solution was replaced with a 12% w/v calcium chloride water/ethanol (75/25 v/v) solution. After four rinses, the calcium concentration was about 0.5-0.6 g/l of moist beads. The beads were highly transparent without any observable surface defects.

When contacted with 5 mM EDTA/50 U pectinase at 25° C., the beads dissolved completely within 5 minutes.

### Example 3

### 1.5% PGA Microcarriers Crosslinked with 3% Calcium

The procedure from example 1 was repeated except that a 1.5% by weight solution of polygalacturonic acid and a pressure of 4 bars were used.

After four rinses, the calcium concentration was about 0.7-0.8 g/l of moist beads. The beads were highly transparent without any observable surface defects.

### Example 4

### 1.5% PGA Microcarriers Crosslinked with 12% Calcium

The procedure from example 3 was repeated except that a 12% w/v calcium chloride water/ethanol (75/25 v/v) solution was used in lieu of the 3% w/v calcium chloride water/ethanol (75/25 v/v) solution.

After four rinses, the calcium concentration was about 0.7-0.8 g/l of moist beads.

### Example 5-a

### 2% PGA Microcarriers Crosslinked with 3% Calcium

The procedure from example 1 was repeated except that a 2% by weight solution of polygalacturonic acid and a pressure of 5 bars were used. The PGA solution was preheated to 30° C. and jetted at 30° C. into the gelling bath, which was maintained at 25° C.

After four rinses, the calcium concentration was about 0.9-1.0 g/l of moist beads. The beads were highly transparent without any observable surface defects.

### Example 5-b

### 3% PGA Microcarriers Crosslinked with 3% Calcium

The procedure from example 1 was repeated except that a 3% by weight solution of polygalacturonic acid and a pressure of 6 bars were used. The PGA solution was preheated to 40° C. and jetted at 40° C. into the gelling bath, which is maintained at 25° C.

After four rinses, the calcium concentration was 1.48 g/l of moist beads. The beads were highly transparent without any observable surface defects.

When contacted with 5 mM EDTA/50 U pectinase at 25° C., the beads dissolved completely within 10 minutes.

### Example 6

### 2% PGA Microcarriers Crosslinked with 12% Calcium

The procedure from example 5-a was repeated except that a 12% w/v calcium chloride water/ethanol (75/25 v/v) solution was used.

After four rinses, the calcium concentration was about 0.9-1.0 g/l of moist beads.

### Example 7

### PGA Beads Coated with 0.1% Gelatin Crosslinked with Glutaraldehyde

A 0.1% porcine skin gelatin solution was prepared by first soaking 0.5 g (type A) porcine skin (Sigma #G1890) in 20 ml of water and then adding 480 ml heated water (60° C.-80° C.).

Ten milliliters PGA beads prepared according to Examples 1-6 were collected by centrifugation to which 10 mL of the porcine gelatin solution was added. The resulting mixture was gently shaken and incubated for 60 min at 25° C. The supernatant was removed and the beads were washed one time in water.

To crosslink the gelatin coating, 100 ml of 0.05% glutaraldehyde solution, prepared from a 25% stock solution (Sigma C5882) was added to the bead bed and incubated for 1 hour at 23° C. under gentle shaking. The beads were subsequently rinsed three times with water and stored at 4° C. in sterile containers.

### Example 8

### PGA Beads Having a Synthemax® II-SC Synthetic Copolymer Surface

About 9 ml of swollen beads prepared according to Example 1 were placed in a 50 ml plastic centrifuge tube. Added to the beads was 36 ml of a 0.25 mg/ml aqueous solution of adhesion promoting peptides to form Corning Incorporated Synthemax® II-SC surface. The tube was gently shaken and left undisturbed for 30 min at 40° C. allowing the synthetic copolymer to functionalize the beads. After cooling the functionalized beads were washed three times with DI water. Beads were stored at 4° C. water in sterile containers.

### Example 9

### Calcium Titration

To quantify the calcium content, 1 ml of PGA beads were digested by combining with 10 ml of a 5 mM EDTA/50 U pectinase solution. The suspension was vigorously shaken and left for one hour at 25° C. under gentle shaking until digestion was completed.

The calcium content of the beads was quantified by Inductively Coupled Plasma Optical Emission Spectrometry (Axial ICP-OES, Varian 720 ES tool). The sample to be analyzed was prepared by diluting 100 μl of the solution containing the digested beads in 9.9 ml of 1% HNO₃. A calibration curve was built using solutions of known calcium concentrations prepared from an aqueous 1 g/l standard solution, which was diluted with HNO₃ as was done for each sample.

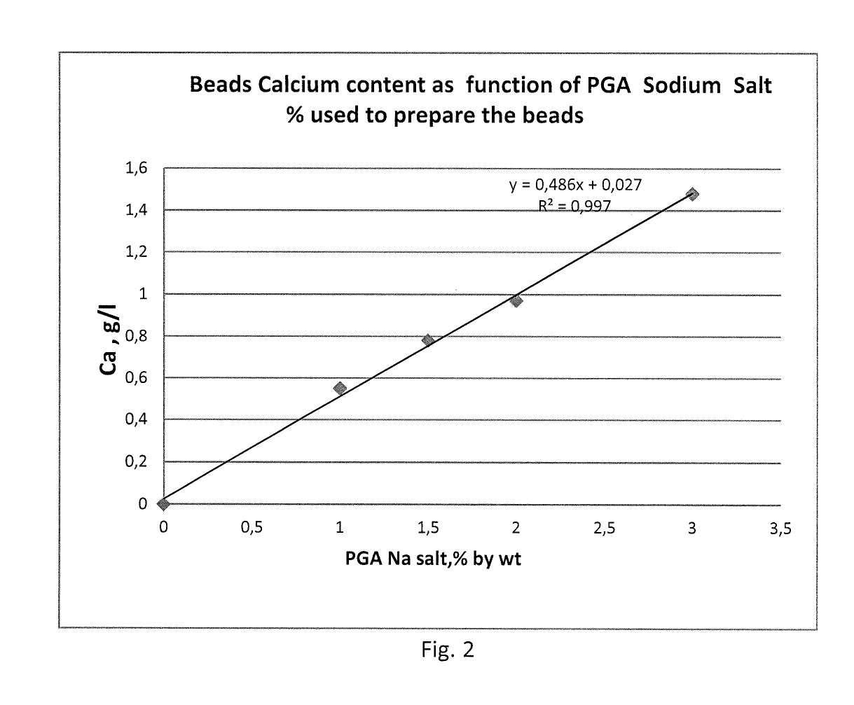

The linear relationship between the calcium content at equilibrium (after extensive washing with water in order to remove unbound calcium) and the amount of PGA sodium salt used to prepare the beads by external gelation is shown in FIG. 2. The calcium content corresponds to the calcium capture capacity of the polygalacturonic acid hydrogel.

### Example 10-a

### hMSC Static Culture with Peptide Copolymer-Coated Microcarriers

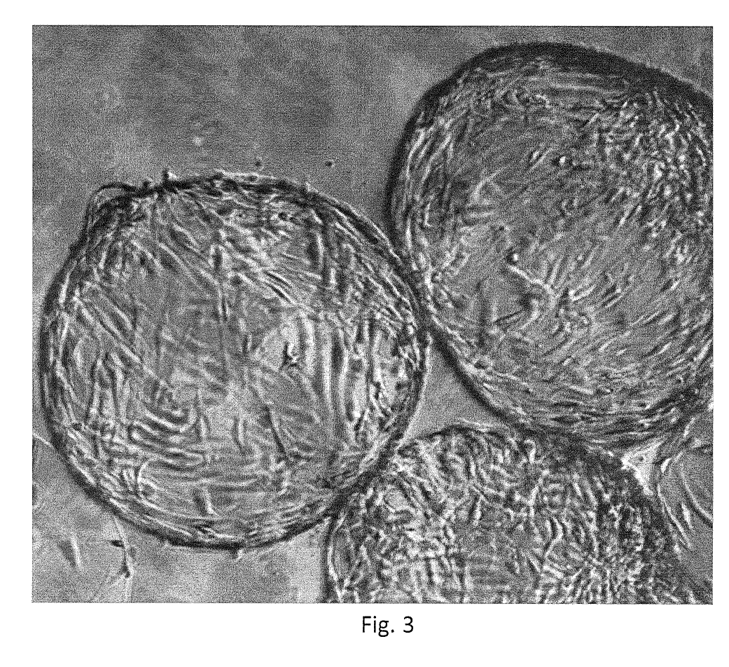

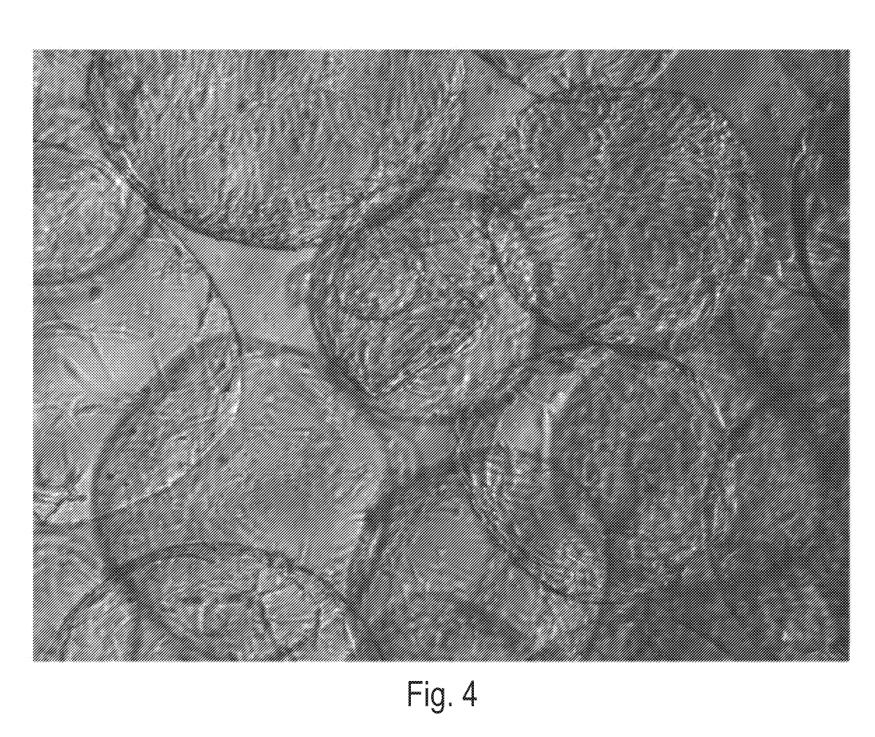

Beads prepared according to Example 2 and provided with a Synthemax® II-SC copolymer surface according to Example 8 were sanitized with 70% ethanol/water and twice rinsed with phosphate buffered saline (dPBS) and then with MesenCult™-XF complete medium. (MC-XF). Bone marrow-derived mesenchymal stem cell (hMSC) culture was carried out under static conditions in MC-XF in 24 well ULA plates. Cells were seeded at 100 k cells/well. Cells were harvested from the microcarriers by treatment with 50 U pectinase/5 mM EDTA for 5 minutes. Cell morphology 2 days after seeding is shown in FIG. 3. Cell morphology 4 days after seeding is shown in FIG. 4.

### Example 10-b

### hMSC Static Culture with Gelatin-Coated Microcarriers

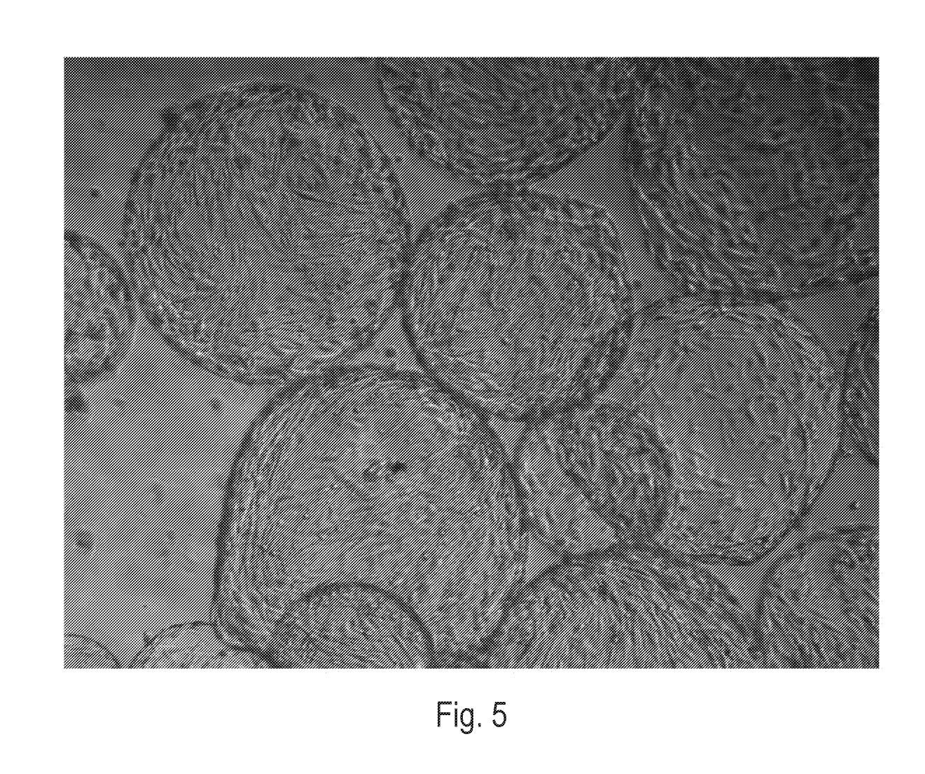

Beads were prepared according to Example 4 and coated with gelatin according to Example 7. FIG. 5 shows an example phase contrast microscopy image of the adhesion and growth of human bone marrow-derived mesenchymal stem cells (hMSC) 2 days after seeding at 100 k cells/well in 24 well plates.

### Example 11

### Expansion of Vero Cells on Gelatin-Coated PGA Microcarriers

Vero cells were cultured under continuous stirring on gelatin-coated, externally crosslinked 1% PGA microcarriers prepared according to Example 2 and coated according to Example 7.

The beads were first sanitized with 70% ethanol/water, twice rinsed with phosphate buffered saline (dPBS), and then rinsed with (IMDM+10% FBS+5 ml penicillin streptomycin+5 ml Glutamax™ media).

Cell culture was performed in Corning Incorporated disposable spinner flasks using IMDM supplemented with 10% FBS+5 ml penicillin streptomycin+5 ml Glutamax™ media as the culture medium. The flasks were seeded with 1M of Vero (p5) without stirring for 2 h, followed by continuous agitation. The passage number designation (p#, in this example “p5”) indicates the number (#) of expansion and harvest cycles used to produce the cells (i.e., the number of divisions the cells have had in culture).

Cell harvesting at day 5 was done with 10 mL of 50 U/ml pectinase, 5 mM EDTA.

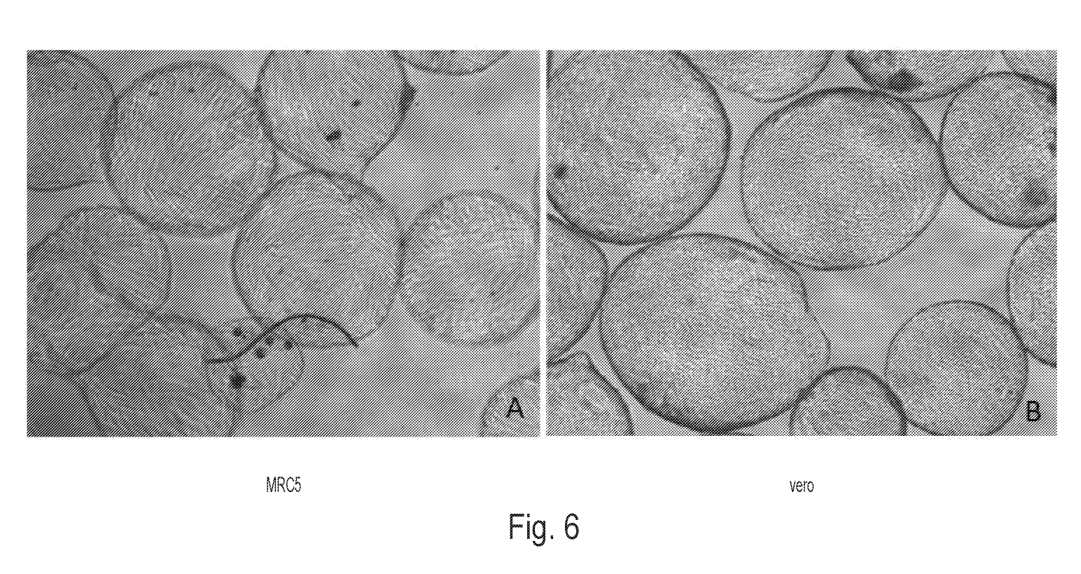

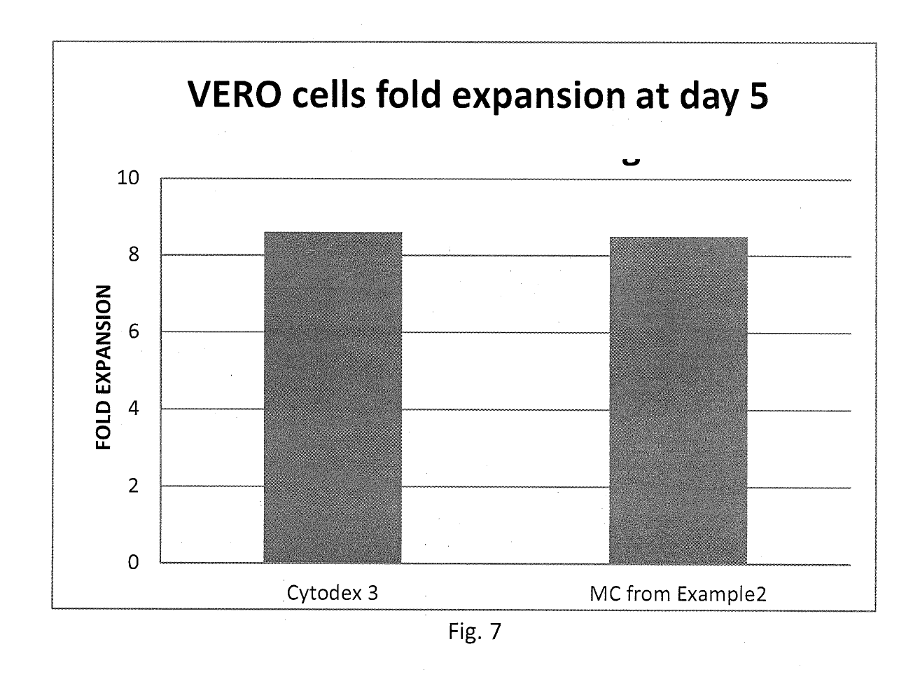

FIG. 6 shows an example phase contrast microscopy image of the adhesion and growth of the Vero cells 4 days after seeding. Fold expansion data is shown in FIG. 7.

### Example 12

### Expansion of MRC5 Cells on Gelatin-Coated PGA Microcarriers

Human fetal lung fibroblast (MRC5) cells were cultured under intermittent stirring on gelatin-coated, externally crosslinked 1% PGA microcarriers prepared according to Example 2 and coated according to Example 7.

The beads were first sanitized with 70% ethanol/water, twice rinsed with phosphate buffered saline (dPBS), and then rinsed with (IMDM+10% FBS+5 ml penicillin streptomycin+5 ml Glutamax™ media).

Cell culture was performed in Corning Incorporated disposable spinner flasks using IMDM supplemented with 10% FBS+5 ml penicillin streptomycin+5 ml Glutamax™ media as the culture medium. The flasks were seeded with 1M of MRC5 cells (p4) without stirring overnight, followed by intermittent agitation (¼ h per 2 h).

Cell harvesting at day 5 was done with 10 mL of 50 U/ml pectinase, 5 mM EDTA.

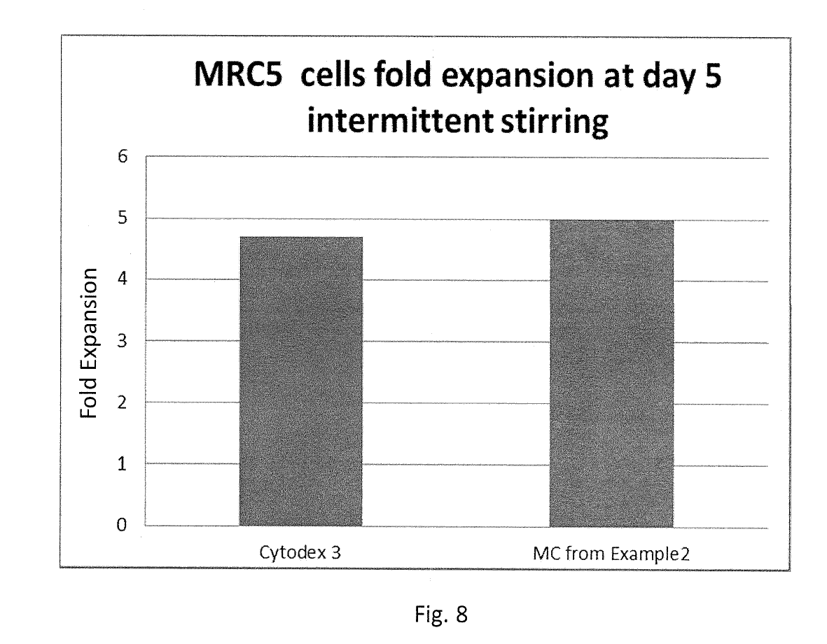

FIG. 6 shows an example phase contrast microscopy image of the adhesion and growth of the MRC5 cells 4 days after seeding. Fold expansion data is shown in FIG. 8.

The micrographs in FIG. 6 show that the Vero and MRC5 cells were able to adhere and reach confluence on the PGA microcarriers.

### Example 13

### Expansion of hMSC Cells on Peptide Copolymer-Coated Microcarriers

hMSC cells were cultured under continuous stirring on externally crosslinked PGA beads prepared as described in Example 1 and provided with a Synthemax® II-SC copolymer surface according to Example 8.

The beads were first sanitized with 70% ethanol/water, twice rinsed with phosphate buffered saline (dPBS), and then with MesenCult™-XF complete medium (MC-XF). Cells were seeded at 1M cells/flask and cell culture was performed in Corning Incorporated disposable spinner flasks using MC-XF.

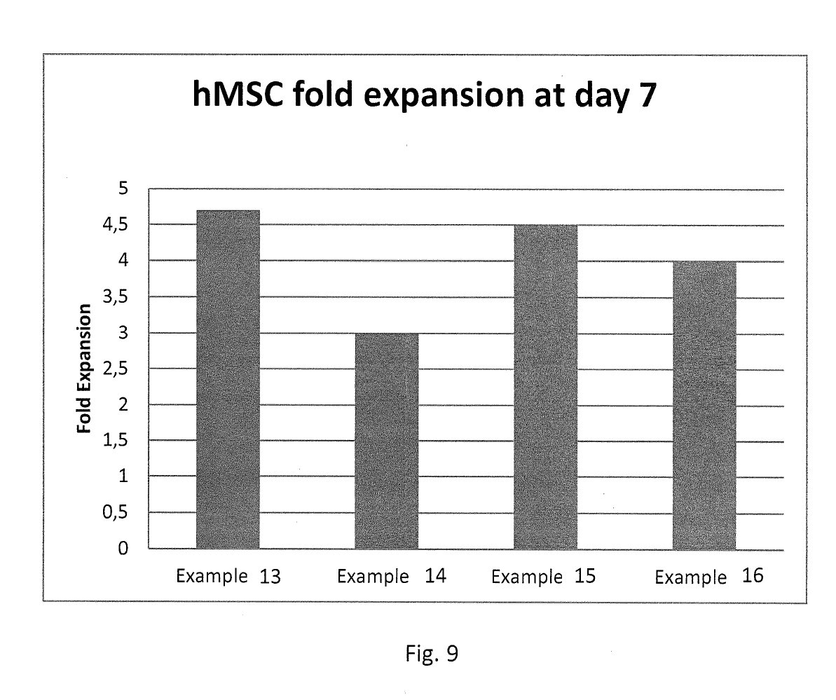

Cells were harvested from the microcarriers at day 7 by treatment with 10 ml 50 U pectinase/5 mM EDTA for 5 minutes. Fold expansion data is shown in FIG. 9.

### Example 14

### Expansion of hMSC Cells on Peptide Copolymer-Coated Microcarriers

Expansion of hMSC cells under continuous stirring as described in Example 13 was repeated except that externally crosslinked PGA beads prepared from a 2% PGA solution prepared as described in Example 5-a and provided with a Corning Synthemax II synthetic peptide copolymer surface as described in Example 8 were used. Fold expansion data is shown in FIG. 9.

### Example 15

### Expansion of hMSC Cells on Gelatin-Coated Microcarriers

hMSC cells were cultured under continuous stirring on externally crosslinked PGA beads prepared as described in Example 1 and coated with gelatin as described in Example 7.

The beads were first sanitized with 70% ethanol/water, twice rinsed with phosphate buffered saline (dPBS), and then with MesenCult™-XF complete medium (MC-XF). Cells were seeded at 1M cells/flask and cell culture was performed in Corning Incorporated disposable spinner flasks using MC-XF.

Cells were harvested from the microcarriers at day 7 by treatment with 10 ml 50 U pectinase/5 mM EDTA for 5 minutes. Fold expansion data is shown in FIG. 9.

### Example 16

### Expansion of hMSC Cells on Gelatin-Coated Microcarriers

Expansion of hMSC cells under continuous stirring conditions as described in Example 15 was repeated except using the externally crosslinked gelatin-coated PGA beads prepared as described in Example 5-a and coated with gelatin as described in Example 7. Fold expansion data is shown in FIG. 9.

### Example 17

### Chemical Stability of PGA Microcarriers

The chemical stability of the microcarrier beads was evaluated by adding 1 ml swollen beads and 5 ml Dulbecco's Phosphate-Buffered Saline (dPBS) (1×) to a plastic centrifuge tube containing. The tube was incubated for 24 hr at 37° C. The volume of the beads after 24 hours was comparable to the initial volume showing that the beads do not dissolve in the phosphate buffer.

### Example 18

Monodisperse microcarrier beads were produced from a 1.5 wt. % PGA solution using an electromagnetically-driven laminar jet nozzle system (Nisco Engineering AG, Zurich, Switzerland). The system is equipped with a 100 μm nozzle. The frequency was set to 2.5 kHz, and the amplitude to 100%. The solution flow rate, which is generated by applying a pressure of about 3 psi, was about 100 ml/h.

The nozzle was positioned about 7.5 cm above the surface of a gelling bath (4 wt. % CaCl₂ solution in 50:50 v/v water/ethanol). The bath was continuously stirred (170 rpm).

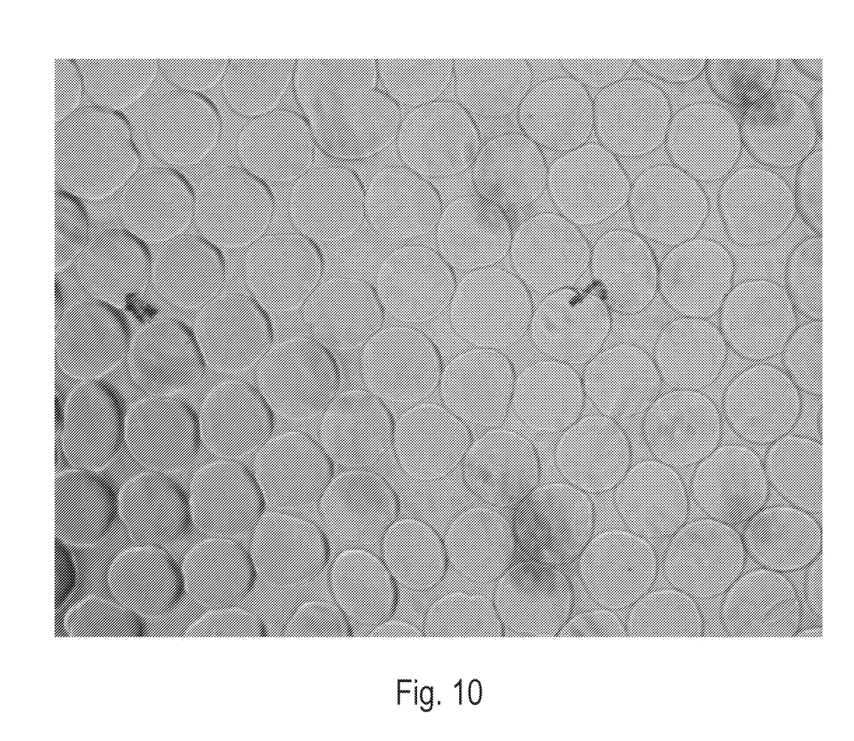

The resulting microbeads had an average diameter of 240±15 μm, which corresponds to a coefficient of variation (CV) of 6.25%. This narrow size distribution is shown in FIG. 10 (magnification: 4×).

The beads were gelatin coated as described in Example 7, except that 50 ml of 0.05% glutaraldehyde solution was used instead of 100 ml to crosslink the gelatin coating.

### Example 10-c.

### MRC5 Static Culture with Gelatin-Coated Microcarriers

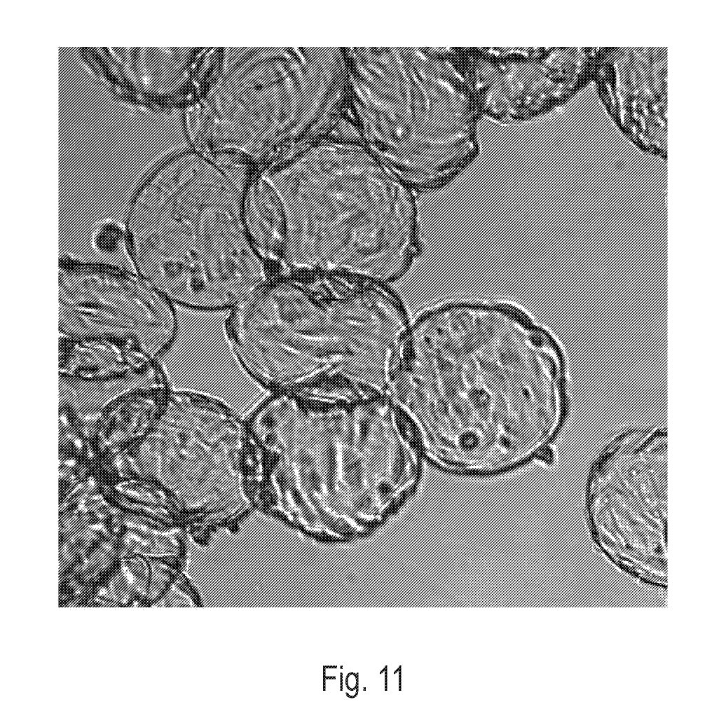

Human fetal lung fibroblast (MRC5) cells were cultured in static conditions on microbeads prepared and coated with gelatin according to Example 18. Cells were seeded at 100 k cells/well in 24 well ULA plates using IMDM supplemented with 10% FBS as the culture medium. Cell morphology 1 day after seeding is shown in the phase contrast microscopy image of FIG. 11.

### Example 19

### Grafting of Vitronectin Peptide to PGA Microcarriers

About 3 ml of swollen beads prepared according to Example 18 were placed in a 15 ml plastic centrifuge tube. Twelve milliliters of an aqueous solution comprising 200 mM N-ethyl-N′-(3-(dimethylamino)propyl)carbodiimide (EDC) and 50 mM N-hydroxysuccinimide (NHS) was added. The tube was gently agitated for 30 min at 23° C. allowing the activation of carboxylic acid groups within the PGA beads. The activated microcarriers were collected by centrifugation and rinsed three times with 10 ml deionized water.

The rinsed microcarriers were re-suspended in 12 ml borate buffer (pH 9.2) containing 49 mg of vitronectin peptide (Ac-Lys-Gly-Pro-Gln-Val-Thr-Arg-Gly-Asp-Val-Phe-Thr-Met-Pro-NH2; catalog number: 341587 available from American peptide). The suspended microcarriers were left to react for 30 minutes under gentle agitation.

The peptide conjugated-microcarriers were collected by centrifugation and washed three times with 10 ml PBS buffer, pH 7.4. Excess activated ester was deactivated by blocking with 12 ml 1M ethanolamine (pH 8.4) for 60 minutes.

The peptide grafted and blocked microcarriers were collected and rinsed three times with PBS. After removing excess PBS, the microcarriers were rinsed 2 times with ethanol/water (70/30 v/v) and stored prior to cell culture at 4° C. in sterile containers.

### Example 10-d

### hMSC Static Culture Using VN-Grafted Microcarriers

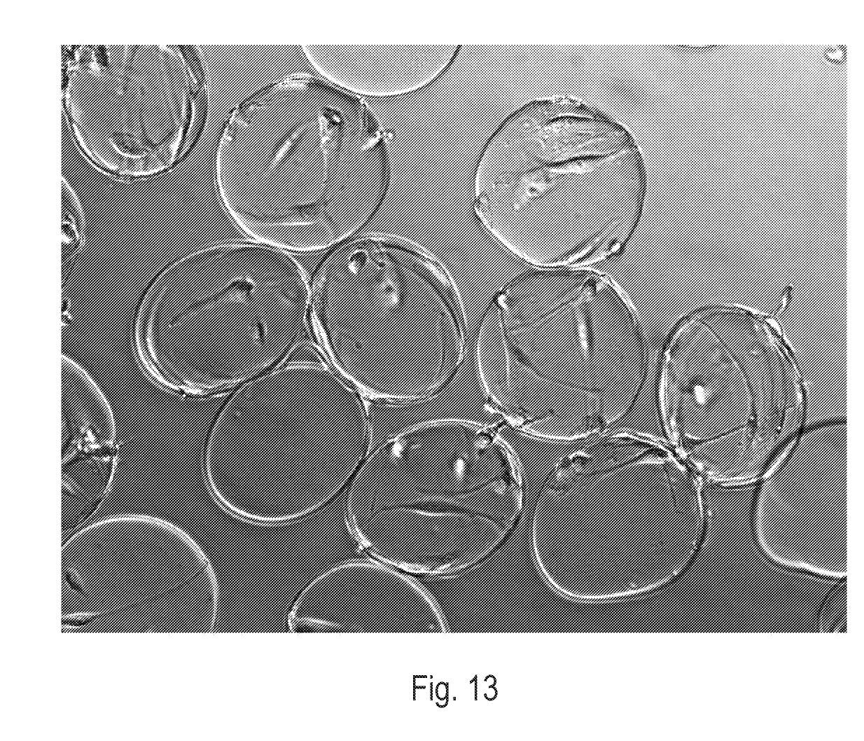

Human mesenchymal stem cells (hMSC 2637, p3) were cultured in static conditions on VN-grafted microcarrier beads prepared according to Example 19. Cells were seeded at 50 k cells/well in 24 well ULA plates. FIG. 13 is a phase contrast image showing the hMSC cells in serum free medium (Mesencult XF) 24 h after seeding.

Data from Examples 10a-10d demonstrate that wholly synthetic microcarriers and gelatin-coated microcarrier each support hMSC attachment and growth.

### Example 20

### Generation of Dissolvable Microcarriers Having Different Sizes Using External Gelation

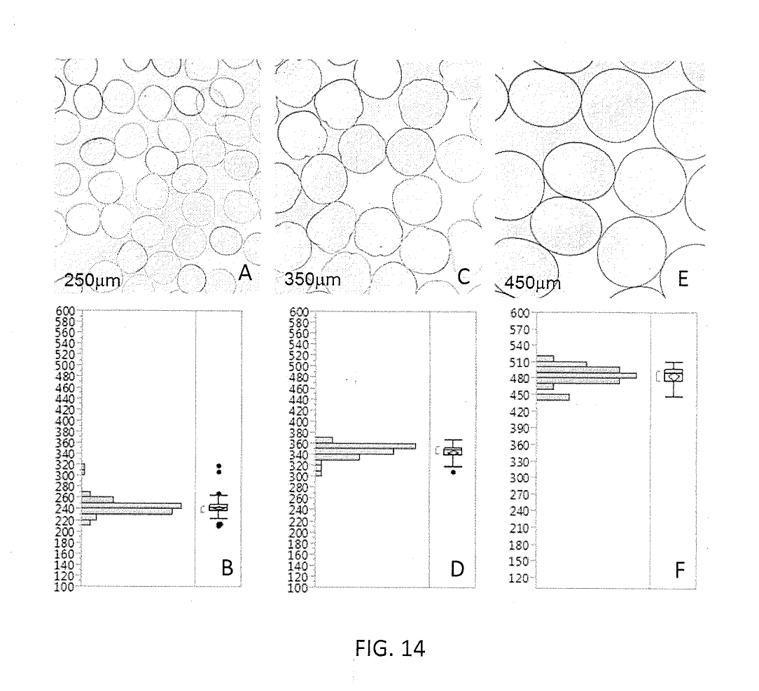

Polygalacturonic acid sodium salt was dissolved 2% in water. External gelation was processed using NISCO single nozzle vibration encapsulator system. The beads were dropped into 4% CaCl₂ dissolved in 1:1 (v/v) mixing of ethanol and water. For targeted size ranges, different sized nozzles, flow rates and vibration frequencies were used according to Table I. FIG. 14 shows the optical microscopy images and size distributions of beads made with the different parameters. The average sizes and coefficients of variation for targeted sizes of 250 μm (shown in FIG. 14A and FIG. 14B), 350 μm (shown in FIG. 14C and FIG. 14D) and 450 μm (shown in FIG. 14E and FIG. 14F) were analyzed using optical imaging.

TABLE I

| Targeted | Nozzle | | | | |
|---|---|---|---|---|---|
| Size | Size | Flow Rate | Frequency | Obtained Size | CV |
| 250 μm | 100 μm | 120 ml/hr | 3.00 kHz | 243 ± 15 μm | 0.062 |
| 350 μm | 100 μm | 120 ml/hr | 1.15 kHz | 346 ± 11 μm | 0.032 |
| 450 μm | 250 μm | 400 ml/hr | 1.15 kHz | 485 ± 17 μm | 0.035 |

### Example 21

### Settling Time Measurement and Analysis

To evaluate settling speed, embodiments of microcarriers of different sizes were compared with three commercially available microcarriers: Cytodex®-1 (cross-linked dextran-based microcarriers commercially available from GE Healthcare Bio-Sciences, Pittsburgh, Pa.), SoloHill® P102-1521 (plastic cross-linked polystyrene microcarriers commercially available from Pall Corporation, Port Washington, N.Y.) and Hillex® II (modified polystyrene microcarriers commercially available from Pall Corporation, Port Washington, N.Y.). These three types of commercially available microcarriers range from hydrogel to solid plastic and have densities ranging from 1.02 to 1.09. Optical Density (OD) was measured, as described in more detail below, and used to determine the concentration of beads in suspension. Microcarriers are able to block visible light due to obscuration. Generally, a lower OD correlates to a lower concentration of microcarriers in suspension.

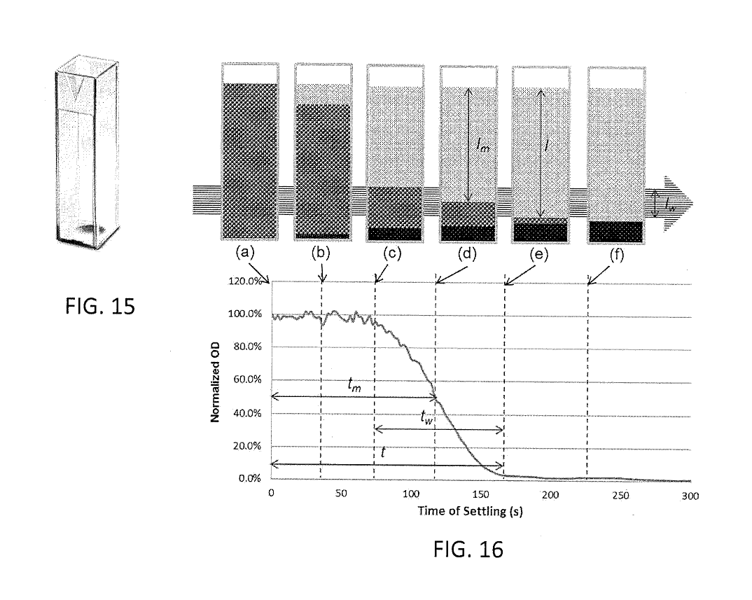

FIG. 16 illustrates an exemplary method used to measure OD. The top portion of FIG. 16 shows schematic images of beads settling in cuvettes at different stages. The wide arrow across the cuvette image illustrates the path of light used in the method for measuring OD. As shown, the light path may be close the bottom of the cuvette. FIG. 15 illustrates an exemplary cuvette. FIG. 16 is an illustration of settling of microcarriers in a cuvette such as the one shown in FIG. 15. The bottom portion of FIG. 16 is a graph illustrating the change in OD over a period of time during which the microcarrier beads settle out of solution. Generally, at the beginning of the measurement, the microcarrier beads are completely suspended in solution as shown in FIG. 16(a) and light is blocked at the highest level. As the microcarrier beads start to settle, the top part of the suspension begins to clear because the microcarrier beads move in the same direction, although the concentration of microcarrier beads in the path of the light remains relatively unchanged, as shown by FIG. 16(b). As the microcarrier beads begin to settle below the upper limit of the path of the light, OD decreases as is shown in FIG. 16(c). When the microcarrier beads reach approximately the middle of the path of the light, as shown in FIG. 16(d), OD is reduced by half. The period of time for the microcarrier beads to reach approximately the middle of the path of the light is represented by tₘ. When the height of total suspension and the position of the path of the light are fixed, the faster the beads settle, the smaller will be. Settling speed, represented herein by vₘ, can be estimated by the time, represented herein by tₘ, for the microcarriers to travel the distance, represented herein by lₘ, from the top of the suspension to the middle of the path of the light. This can be shown in Formula (1):

$${v}_{m} = \frac{{l}_{m}}{{t}_{m}} \qquad ( 1 )$$

For a population of microcarrier beads with non-uniform size, different settling speeds are expected for different sized microcarrier beads. As such, settling speed vₘ represents a medium settling speed of the population.

The path of the light has a width, represented herein by l_{w}, and the time, represented herein by t_{w}, for the microcarrier beads to travel the width l_{w} is shown by the progression from FIG. 16(c) to FIG. 16(e). When the microcarrier bead population has a uniform settling speed (i.e.: the microcarrier beads have a uniform size distribution), t_{w} can be estimated using the width of the path of the light l_{w} and settling speed vₘ as shown in Formula (2):

$${t}_{w} = \frac{{l}_{w}}{{v}_{m}} \qquad ( 2 )$$

When the microcarrier bead population has a distribution of different settling speeds (i.e.: the microcarrier beads have a non-uniform size distribution), the fastest settling microcarrier beads will reach the path of the light sooner than the slowest settling microcarrier beads. As the fastest settling microcarrier beads pass through the path of the light, a reduction of OD is observed, however, not until the slowest settling microcarrier beads pass through the path of the light is a complete reduction of OD observed. A microcarrier bead population having a distribution of different settling speeds will exhibit a longer t_{w} than a microcarrier bead population having a uniform settling speed. As such, the shorter the t_{w}, the more uniform the settling speed of the population of the microcarrier beads and the more uniform the size distribution of the population of the microcarrier. While the above assumes that a starting point and an ending point of a change in OD can be determined, it should be understood that such starting and ending points may be difficult to define. As such, the slope of the change of OD can be used to represent the magnitude of the variation of the settling speeds in a microcarrier bead population.

In practice, it is preferable to wait for all the beads to completely separate from the supernatant in order to conclude the settling process. Therefore the time, represented herein by t, to observe a complete reduction of OD is more relevant to estimate final settling time. Final settling time may be determined using both the average settling time of a microcarrier bead population and the variation is of the the settling times of the microcarrier bead population. For quantitative measurement, final settling time may be determined by using tₘ and t_{w} or using the slope of the change of OD.

In the present experiment, the three types of commercially available microcarriers were rehydrated and suspended in DPBS solution. Dissolvable microcarriers (DMCs) with three different sizes (250 μm, 350 μm and 450 μm) were formed as described in Experiment 1. About 0.5 ml of packed microcarriers was added into each cuvette. Then, DPBS was filled into the cuvettes to a total volume of 3.5 ml. Immediately before measurement, the microcarriers were suspended by pipetting up and down 10 times. OD was measured in accordance with the method described above and was measured at a wavelength of 400 nm. Other visible wavelengths can be chosen as well. OD measurements were performed every 2.0 seconds. Because the various types of microcarriers are formed from different materials, having different optical indexes, are different sizes and have different optical clarities, Initial OD was used to normalize the measurement of each sample so that the different samples could be compared.

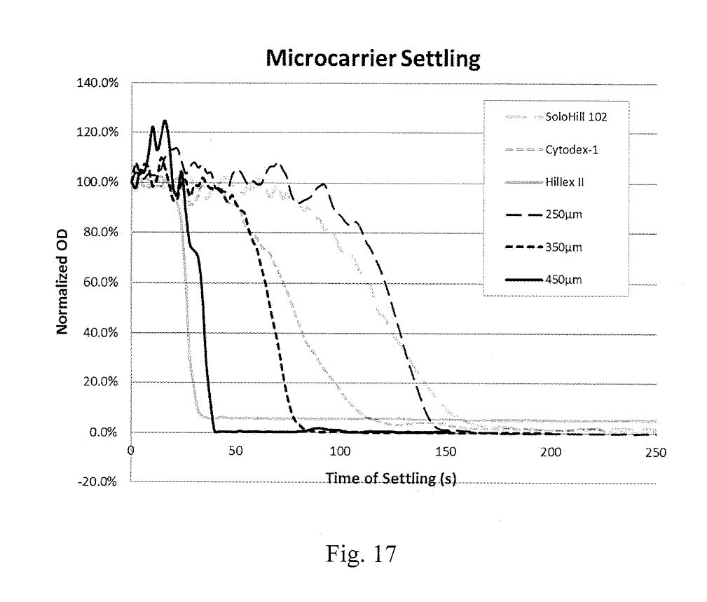

The settling speed measurement results are shown in FIG. 17. The three sizes of DMCs were compared with the three commercially available microcarriers. The results showed that DMCs of 350 μm diameter settled 2 times as fast as the DMCs of 250 μm, and DMCs of 450 μm diameter settled 3 times as fast as the DMCs of 250 μm. By changing the size of the DMCs, the settling speeds were able to match the medium settling speeds of the three commercial beads made of different materials and with different densities. As compared with the Cytodex®-1 and SoloHill® P102-1521 microcarriers, the DMCs having sizes of 250 μm and 350 μm demonstrated comparable medium settling speeds, but demonstrated much shorter t_{w} and steeper slopes of change of OD. As will be discussed in more detail in Example 21, the shorter t_{w} and steeper slopes of change of OD is the result of a smaller size distribution than the commercially available microcarriers which provides a more consistent settling speed as compared with the commercially available microcarriers. Comparing settling time t, DMCs having sizes of 250 μm and 350 μm settle quicker than Cytodex®-1 and SoloHill® P102-1521 microcarriers, which suggests that DMCs will need much shorter time to complete settling compared to the commercially available microcarriers.

### Example 21

### Microcarrier Size Distribution and Radius of Curvature Analysis

The size distribution and radius of curvature of dissolvable microcarriers (DMCs) formed in accordance with the microcarriers having a targeted size of 250 ml in Example 20 were compared to three commercially available microcarriers: Cytodex®-1, SoloHill® P102-1521 and Cytodex®-3 (cross-linked dextran-based microcarriers commercially available from GE Healthcare Bio-Sciences, Pittsburgh, Pa.). Microcarrier size was determined using known optical microscopy techniques and software to analyze images captured with an optical microscope. Radius of curvature was then calculated from the determined microcarrier size using Formula (3):

$$\vert K \vert = \frac{1}{R} \qquad ( 3 )$$

where K is microcarrier radius of curvature and R is microcarrier radius. For each type of microcarrier, Table II shows size range d5-d95 (where d5 is the microcarrier diameter that is larger than the diameters of 5% of the microcarrier population and d95 is the microcarrier diameter that is larger than the diameters of 95% of the microcarrier population), size range d10-d90 (where d10 is the microcarrier diameter that is larger than the diameters of 10% of the microcarrier population and d90 is the microcarrier diameter that is larger than the diameters of 95% of the microcarrier population), size spread Δd5-d95 (the difference between d95 and d5), the coefficient of variation for d5-d95, size spread Δd10-d90 (the difference between d90 and d10) and the coefficient of variation for d10-d90.

TABLE II

| | Cytodex ®-1 | Cytodex ®-3 | SoloHill ® | DMC |
|---|---|---|---|---|
| d5-d95 | 202-290 μm | 165-236 μm | 134-193 μm | 233-256 μm |
| d10-d90 | 210-279 μm | 174-230 μm | 138-188 μm | 234-252 μm |
| Δd5-d95 | 88 μm | 71 μm | 59 μm | 23 μm |
| d5-d95 | 0.179 | 0.177 | 0.180 | 0.047 |
| CV | | | | |
| Δd10-d90 | 69 μm | 56 μm | 50 μm | 18 μm |
| d10-d90 | 0.141 | 0.139 | 0.153 | 0.037 |
| CV | | | | |

For each type of microcarrier, Table III shows average microcarrier diameter (d), average radius of curvature (K), radius of curvature at d5, radius of curvature at d95, and radius of curvature spread from d5 to d95 (the difference between d5 radius of curvature and d95 radius of curvature).

TABLE III

| | Cytodex ®-1 | Cytodex ®-3 | SoloHill ® | DMC |
|---|---|---|---|---|
| Avg. diameter (μm) | 244 | 201 | 161 | 240 |
| Avg. K (cm⁻¹) | 82.0 | 99.5 | 124.2 | 83.3 |
| K at d5 (cm⁻¹) | 99.0 | 121.2 | 149.3 | 85.8 |
| K at d95 (cm⁻¹) | 69.0 | 84.7 | 103.6 | 78.1 |
| ΔK_{d5} − K_{d95} (cm⁻¹) | 30.0 | 36.5 | 45.6 | 7.7 |

The data in Tables II and III shows that DMCs as disclosed herein have a more uniform size distribution and a more uniform radius of curvature than the three commercially available microcarriers. As previously described, a uniform size distribution enables settling of beads at a consistent speed which allows for more predictable separation of microcarriers from supernatant during medium exchange or culture produce isolation. A uniform size distribution and a uniform radius of curvature also provides the same surface area available for cells to seed per microcarrier which enables cells to reach confluence at the same, or at a similar, time.

### Example 22

### Microcarrier Particle Count Analysis

Debris particle count resulting from dissolvable microcarriers (DMCs) formed in accordance with microcarriers described in Example 20 was compared to two commercially available microcarriers: SoloHill® P102-1521 and Cytodex®-3. Multiple steps of washing each type of microcarrier were performed prior to stirring to reduce the number of particles to less than 2 particles per mL. For each type of microcarrier, a volume of microcarriers of about 1000 cm² was placed in separate Corning® 125 mL Disposable Spinner Flasks (commercially available from Corning, Inc., Corning, N.Y.) and suspended in about 100 mL Dulbecco's phosphate-buffered saline (DPBS). Two Disposable Spinner Flasks were filled with DMCs. The microcarriers were continuously stirred at a speed of about 60 rpm at room temperature for a total of 6 days. The DMCs in one of the Disposable Spinner Flasks were dissolved in accordance with methods described herein, and the DMCs in the other of the Disposable Spinner Flasks were not dissolved. After stirring was complete, the microcarriers were separated from the DPBS and the particle count in the DPBS was measured with an HIAC 9703+ Particle Counter (commercially available from Beckman Coulter Life Sciences, Indianapolis, Ind.) using the light obscuration particle count test as described in The United States Pharmacopeia and The National Formulary Section 788 (USP<788>) entitled “Particulate Matter in Injections”. As indicated in USP<788> a preparation complies with the test if the average number of particles present in the units tested does not exceed 25 particles per mL equal to or greater than 10 μm and does not exceed 3 particles per mL equal to or greater than 25 μm. Table IV shows the number of particles remaining having a size of greater than or equal to 25 μm per mL of DPBS for each of the microcarriers, including non-dissolved DMC and dissolved DMC. Table V shows the remaining number of particles having a size of greater than or equal to 10 μm per mL of DPBS for each of the microcarriers, including non-dissolved DMC and dissolved DMC.

TABLE IV

| | # of Particles ≥ 25 μm per mL |
|---|---|
| Cytodex ®-3 | 0.4 |
| SoloHill ® | 1.0 |
| DMC (non-dissolved) | 0.3 |
| DMC (dissolved) | 0.1 |

TABLE V

| | # of Particles ≥ 10 μm per mL |
|---|---|
| Cytodex ®-3 | 26 |
| SoloHill ® | 17 |
| DMC (non-dissolved) | 7 |
| DMC (dissolved) | 9 |

As shown in Tables IV and V, where the DMCs were both dissolved and non-dissolved, fewer DMC particles remained after stirring in the Disposable Spinner Flasks than remained after stirring the other two commercially available microcarriers. This was true for particles having a size of greater than 10 μm and for particles having a size of greater than 25 μm.

### Comparative Example 1

Vero cell culture was repeated as described in Example 11 except that non-digestible Cytodex®-3, substrates area used instead of the PGA microcarriers. Trypsin was needed to detach the cells from the surface of the Cytodex®-3 beads. Fold expansion data is summarized in FIG. 7. The expansion obtained with the digestible microcarriers is comparable to the expansion on Cytodex®-3.

### Comparative Example 2

MRC5 cell culture was repeated as described in Example 12 except that non-digestible Cytodex®-3 substrates are used instead of the PGA microcarriers. Trypsin was needed to detach the cells from the surface of the Cytodex® beads. Fold expansion data is summarized in FIG. 8. The expansion obtained with the digestible microcarriers is comparable to the expansion on Cytodex®-3.

### Comparative Example 3

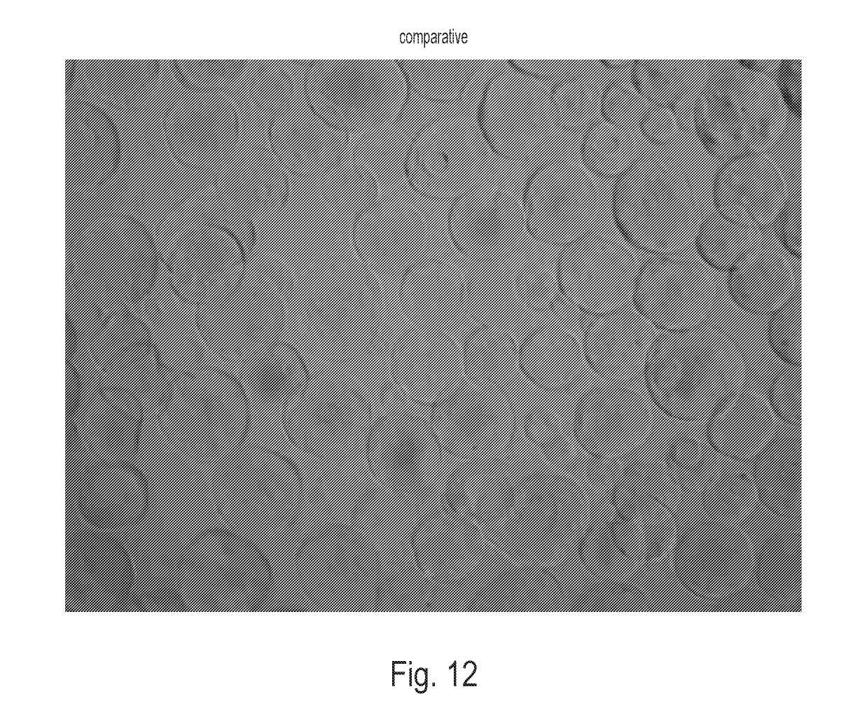

FIG. 12 is a phase contrast microscopy image of beads formed via internal gelation according to Example 1 of WO2014/209865. The beads have an average diameter of 231±54 μm, which corresponds to a coefficient of variation (CV) of 23%.

The disclosed methods provide an inexpensive and environmentally-friendly route for the preparation of highly-transparent PGA microcarriers that are free of undesired inclusions and surface defects and which support non-proteolytic cell separation and harvesting.

According to an aspect (1) of the present disclosure, a cell culture article is provided. The cell culture article comprises a substrate comprising a polygalacturonic acid compound selected from at least one of: pectic acid or salts thereof, and partially esterified pectic acid having a degree of esterification from 1 to 40 mol % or salts thereof, wherein the polygalacturonic acid compound is crosslinked with a divalent cation and the divalent cation concentration ranges from 0.5 to 2 g/l of the substrate.

According to another aspect (2) of the present disclosure, the article according to aspect (1) is provided wherein the substrate is spherical or substantially spherical.

According to another aspect (3) of the present disclosure, the article according to aspect (2) is provided wherein the substrate comprises a diameter of 10 to 500 micrometers.

According to another aspect (4) of the present disclosure, the article according to any of aspects (1)-(3) is provided wherein a plurality of the cell culture articles comprise a coefficient of variation of less than 20%.

According to another aspect (5) of the present disclosure, the article according to any of aspects (1)-(4) is provided wherein a plurality of the cell culture articles comprise a coefficient of variation of less than 10%.

According to another aspect (6) of the present disclosure, the article according to any of aspects (2)-(5) is provided wherein a plurality of the cell culture articles comprise size spread Δd5-d95 of less than 25 micrometers, wherein d5 is a diameter that is larger than the diameters of 5% of the plurality of the cell culture articles, wherein d95 is a diameter that is larger than the diameters of 95% of the plurality of the cell culture articles, and wherein Δd5-d95 is the difference between d95 and d5.

According to another aspect (7) of the present disclosure, the article according to any of aspects (2)-(6) is provided wherein a plurality of the cell culture articles comprise size spread Δd10-d90 of less than 20 micrometers, wherein d10 is a diameter that is larger than the diameters of 10% of the plurality of the cell culture articles, wherein d90 is a diameter that is larger than the diameters of 90% of the plurality of the cell culture articles, and wherein Δd10-d90 is the difference between d90 and d10.

According to another aspect (8) of the present disclosure, the article according to any of aspects (2)-(7) is provided wherein a plurality of the cell culture articles comprise a radius of curvature spread ΔK_{d5}-K_{d95} of less than 10 cm⁻¹, wherein K_{d5} at is the radius of curvature of a cell culture articles having a diameter that is larger than the diameters of 5% of the plurality of the cell culture articles, wherein K_{d95} is the radius of curvature of a cell culture articles having a diameter that is larger than the diameters of 95% of the plurality of the cell culture articles, and wherein ΔK_{d5}-K_{d95} is the difference between K_{d5} and K_{d95}.

According to another aspect (9) of the present disclosure, the article according to any of aspects (1)-(8) is provided wherein the divalent cation is selected from the group consisting of calcium, magnesium and barium.

According to another aspect (10) of the present disclosure, the article according to any of aspects (1)-(9) is provided further comprising an adhesion polymer on the surface of the substrate.

According to another aspect (11) of the present disclosure, the article according to aspect (10) is provided wherein the adhesion polymer comprises a polypeptide.

According to another aspect (12) of the present disclosure, the article according to any of aspects (10)-(11) is provided wherein the adhesion polymer is grafted to or coated on the surface of the substrate.

According to another aspect (13) of the present disclosure, the article according to any of aspects (1)-(12) is provided wherein the substrate is size-controlled.

According to another aspect (14) of the present disclosure, a method of making a cell culture article is provided. The method comprises dispensing a hydrocolloid solution into a gelation bath, wherein the hydrocolloid solution comprises a polygalacturonic acid compound selected from at least one of: pectic acid or salts thereof, and partially esterified pectic acid having a degree of esterification from 1 to 40 mol % or salts thereof, and wherein the gelation bath comprises a divalent metal salt.

According to another aspect (15) of the present disclosure, the method of aspect (14) is provided wherein the divalent metal is selected from the group consisting of calcium, magnesium and barium.

According to another aspect (16) of the present disclosure, the method of any of aspects (14)-(15) is provided wherein the hydrocolloid solution is dispensed dropwise into the gelation bath.

According to another aspect (17) of the present disclosure, the method of any of aspects (14)-(16) is provided wherein dispensing a hydrocolloid solution into a gelation bath comprises extrusion with a syringe.

According to another aspect (18) of the present disclosure, the method of any of aspects (14)-(17) is provided wherein the hydrocolloid solution comprises 0.5 to 5 wt. % polygalacturonic acid.

According to another aspect (19) of the present disclosure, the method of any of aspects (14)-(18) is provided wherein the gelation bath comprises from 1 to 20% (w/v) divalent metal salt.

According to another aspect (20) of the present disclosure, the method of any of aspects (14)-(19) is provided wherein the gelation bath further comprises an alcohol.

According to another aspect (21) of the present disclosure, the method of any of aspects (14)-(20) is provided further comprising suspending the cell culture article in a peptide-containing solution.

According to another aspect (22) of the present disclosure, a method for culturing cells is provided. The method comprises contacting cells with a cell culture medium having the cell culture article according to any of aspects (1)-(13), and culturing the cells in the medium.

According to another aspect (23) of the present disclosure, a method for harvesting cultured cells is provided. The method comprises culturing cells on the surface of the cell culture article according to any of aspects (1)-(13), and contacting the cultured cells with a mixture of pectinase and a chelator to separate the cells from the cell culture article.

According to another aspect (24) of the present disclosure, the method of aspect (23) is provided wherein the chelator comprises EDTA.

According to another aspect (25) of the present disclosure, the method of any of aspects (23)-(24) is provided wherein contacting the cultured cells with a mixture of pectinase and a chelator to separate the cells from the cell culture article is free of protease.

As used herein, the singular forms “a,” “an” and “the” include plural referents unless the context clearly dictates otherwise. Thus, for example, reference to a “divalent cation” includes examples having two or more such “divalent cations” unless the context clearly indicates otherwise

The term “include” or “includes” means encompassing but not limited to, that is, inclusive and not exclusive.

“Optional” or “optionally” means that the subsequently described event, circumstance, or component, can or cannot occur, and that the description includes instances where the event, circumstance, or component, occurs and instances where it does not.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a hydrocolloid solution comprising pectic acid and water include embodiments where a hydrocolloid solution consists of pectic acid and water and embodiments where a hydrocolloid solution consists essentially of pectic acid and water.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A cell culture article, comprising:
a substrate comprising a polygalacturonic acid compound selected from at least one of:
pectic acid or salts thereof, and
partially esterified pectic acid having a degree of esterification from 1 to 40 mol % or salts thereof,
wherein the polygalacturonic acid compound is crosslinked with a divalent cation and the divalent cation concentration ranges from 0.5 to 2 g/l of the substrate.

2. The article of claim 1, wherein the substrate is spherical or substantially spherical.

3. The article of claim 2, wherein the substrate comprises a diameter of 10 to 500 micrometers.

4. The article of claim 1, wherein a plurality of the cell culture articles comprise a coefficient of variation of less than 20%.

5. The article of claim 1, wherein a plurality of the cell culture articles comprise a coefficient of variation of less than 10%.

6. The article of claim 2, wherein a plurality of the cell culture articles comprise size spread Δd5-d95 of less than 25 micrometers, wherein d5 is a diameter that is larger than the diameters of 5% of the plurality of the cell culture articles, wherein d95 is a diameter that is larger than the diameters of 95% of the plurality of the cell culture articles, and wherein Δd5-d95 is the difference between d95 and d5.

7. The article of claim 2, wherein a plurality of the cell culture articles comprise size spread Δd10-d90 of less than 20 micrometers, wherein d10 is a diameter that is larger than the diameters of 10% of the plurality of the cell culture articles, wherein d90 is a diameter that is larger than the diameters of 90% of the plurality of the cell culture articles, and wherein Δd10-d90 is the difference between d90 and d10.

8. The article of claim 2, wherein a plurality of the cell culture articles comprise a radius of curvature spread ΔKd5-Kd95 of less than 10 cm−1, wherein Kd5 at is the radius of curvature of a cell culture articles having a diameter that is larger than the diameters of 5% of the plurality of the cell culture articles, wherein Kd95 is the radius of curvature of a cell culture articles having a diameter that is larger than the diameters of 95% of the plurality of the cell culture articles, and wherein ΔKd5-Kd95 is the difference between Kd5 and Kd95.

9. The article of claim 1, wherein the divalent cation is selected from the group consisting of calcium, magnesium and barium.

10. The article of claim 1, further comprising an adhesion polymer on the surface of the substrate.

11. The article of claim 10, wherein the adhesion polymer comprises a polypeptide.

12. The article of claim 10, wherein the adhesion polymer is grafted to or coated on the surface of the substrate.

13. The article of claim 1, wherein the substrate is size-controlled.

14. A method of making a cell culture article, comprising:
dispensing a hydrocolloid solution into a gelation bath,
wherein the hydrocolloid solution comprises a polygalacturonic acid compound selected from at least one of:
pectic acid or salts thereof, and
partially esterified pectic acid having a degree of esterification from 1 to 40 mol % or salts thereof, and
wherein the gelation bath comprises a divalent metal salt.

15. The method according to claim 14, wherein the divalent metal is selected from the group consisting of calcium, magnesium and barium.

16. The method according to claim 14, wherein the hydrocolloid solution is dispensed dropwise into the gelation bath.

17. The method according to claim 14, wherein dispensing a hydrocolloid solution into a gelation bath comprises extrusion with a syringe.

18. The method according to claim 14, wherein the hydrocolloid solution comprises 0.5 to 5 wt. % polygalacturonic acid.

19. The method according to claim 14, wherein the gelation bath comprises from 1 to 20% (w/v) divalent metal salt.

20. The method according to claim 14, wherein the gelation bath further comprises an alcohol.

21. The method according to claim 14, further comprising suspending the cell culture article in a peptide-containing solution.

22. A method for culturing cells, the method comprising contacting cells with a cell culture medium having the cell culture article according to claim 1, and culturing the cells in the medium.

23. A method for harvesting cultured cells, the method comprising:
culturing cells on the surface of the cell culture article of claim 1; and
contacting the cultured cells with a mixture of pectinase and a chelator to separate the cells from the cell culture article.

24. The method of claim 23, wherein the chelator comprises EDTA.

25. The method of claim 23, wherein contacting the cultured cells with a mixture of pectinase and a chelator to separate the cells from the cell culture article is free of protease.


SEQUENCE LISTING (2 sequences)

SEQ ID NO: 1 - protein, 14 aa, Unknown
  synthetic peptide
  MOD_RES (1)..(1): ACETYLATION
  MOD_RES (14)..(14): AMIDATION
KGPQVTRGDVFTMP

SEQ ID NO: 2 - protein, 15 aa, Unknown
  synthetic peptide
  MOD_RES (1)..(1): ACETYLATION
  MOD_RES (15)..(15): AMIDATION
KGGNGEPRGDTYRAY